(19) United States
(12) Patent Application Publication
(10) Pub. No.: US 2017/0129966 A1
(43) Pub. Date: May 11, 2017

(54) PRODUCTION OF FC FRAGMENTS

(71) Applicant: Laboratoire Français du Fractionnement et des Biotechnologies, Les Ulis (FR)

(72) Inventor: Nicholas C. Masiello, Uxbridge, MA (US)

(73) Assignee: Laboratoire Français du Fractionnement et des Biotechnologies, Les Ulis (FR)

(21) Appl. No.: 15/315,809

(22) PCT Filed: Jun. 2, 2015
(86) PCT No.: PCT/IB15/01643

§ 371 Date: Dec. 2, 2016

Related U.S. Application Data
(60) Provisional application No. 62/006,584, filed on Jun. 2, 2014.

(51) Int. Cl. C07K 16/32; A01K 67/027; B01D 15/42; B01D 15/30; B01D 61/14; B01D 15/38; C07K 16/04; C12P 21/02

(52) U.S. Cl. CPC C07K 16/32; C07K 16/04; A01K 67/0278; C12P 21/02; C12Y 204/99; C12Y 304/22002; B01D 15/30; B01D 61/145; B01D 15/3809; B01D 15/426; C07K 2317/12; C07K 2317/52; C07K 2317/14; A01K 2227/10; A01K 2217/072; A01K 2267/01; A01K 2207/15; B01D 2311/2626

(57) ABSTRACT

In one aspect, the disclosure provides cells and transgenic non-human mammals for the production of Fc fragments, as well as compositions and uses thereof.

### RELATED APPLICATIONS

This application claims the benefit under 35 U.S.C. §119(e) of U.S. provisional application No. 62/006,584, filed Jun. 2, 2014, which is incorporated by reference herein in its entirety.

### FIELD OF THE INVENTION

The disclosure relates, at least in part, to methods for producing Fc fragments.

### BACKGROUND OF THE INVENTION

Production of therapeutic molecules can involve recombinant expression in cell culture, transgenic expression in animals, and extraction from natural sources. To ensure safety of these molecules for administration to subjects, the therapeutic molecules are purified to remove any impurities or potentially harmful contaminants.

### SUMMARY OF THE INVENTION

Described herein are novel methods of producing Fc fragments. In some aspects of the present disclosure, methods of producing a fragment crystallizable (Fc) fragment are provided, comprising providing a transgenic non-human mammal that has been modified to express an antibody comprising an Fc fragment in the mammary gland; harvesting the antibody comprising an Fc fragment from milk produced by the mammary gland of the transgenic mammal; and isolating the Fc fragment from the antibody.

In another aspect, methods of producing an Fc fragment are provided, comprising providing a mammary epithelial cell that has been modified to express an antibody comprising an Fc fragment; harvesting the antibody comprising the Fc fragment from the mammary epithelial cell; and isolating the Fc fragment from the antibody.

In another aspect, methods of producing an Fc fragment are provided, comprising providing a transgenic non-human mammal that has been modified to express an Fc fragment in the mammary gland; harvesting the Fc fragment from milk produced by the mammary gland of the transgenic mammal; and isolating the Fc fragment.

In yet another aspect, methods of producing an Fc fragment are provided, comprising providing a mammary epithelial cell that has been modified to express an Fc fragment; harvesting the Fc fragment from the mammary epithelial cell; and isolating the Fc fragment.

In some embodiments, isolating the Fc fragment comprises subjecting the antibody sequentially to (a) hydrophobic interaction chromatography; and (b) ultrafiltration. In some embodiments, isolating the Fc fragment comprises subjecting the Fc fragment sequentially to (a) hydrophobic interaction chromatography; and (b) ultrafiltration. In some embodiments, the ultrafiltration is performed in a solution comprising phosphate, NaCl and Tween 80, wherein the phosphate has a concentration between 10 and 100 mM, the NaCl has a concentration between 100 and 500 mM, and the Tween 80 has a concentration between 0 to 0.01%, optionally wherein the solution comprises 20 mM phosphate pH 7.0, 150 mM NaCl and 0.01% Tween 80.

In one embodiment of any of the methods provided, isolating the Fc fragment from the antibody comprises (a) obtaining an antibody comprising an Fc fragment and one or more additional fragments; (b) digesting the antibody of (a) to produce an Fc fragment and one or more additional fragments; (c) separating the Fc fragment from the one or more additional fragments by hydrophobic interaction chromatography, comprising applying the Fc fragment and the one or more additional fragments of (b) to a hydrophobic interaction chromatography column; and recovering the Fc fragment from the hydrophobic interaction chromatography column; and (d) further purifying the recovered Fc fragment by ultrafiltration.

In some embodiments, the one or more additional fragments include a fragment antigen-binding (Fab) fragment, a Fab′ fragment, a F(ab′)2 fragment or a single-chain variable (scFv) fragment.

In another embodiment of any of the methods provided, isolating the Fc fragment comprises (a) obtaining an Fc fragment; (b) subjecting the fragment to hydrophobic interaction chromatography, comprising applying the Fc fragment of (a) to a hydrophobic interaction chromatography column; and recovering the Fc fragment from the hydrophobic interaction chromatography column; and (c) further purifying the recovered Fc fragment by ultrafiltration.

In some embodiments, the mammary epithelial cell is of a non-human mammal. In some embodiments, the non-human mammal is a goat, sheep, bison, camel, cow, pig, rabbit, buffalo, horse, rat, mouse, or llama. In some embodiments, the transgenic non-human mammal is also transgenic for the expression of a sialyl transferase.

In some embodiments, obtaining the antibody comprises purifying the antibody. In some embodiments, the antibody is purified using affinity chromatography. In some embodiments, obtaining the Fc fragment comprises purifying the Fc fragment. In some embodiments, the Fc fragment is purified using affinity chromatography. In some embodiments, the affinity chromatography comprises Protein A affinity chromatography.

In another aspect, methods include subjecting an antibody comprising an Fc fragment sequentially to (a) hydrophobic interaction chromatography; and (b) ultrafiltration, wherein the ultrafiltration is performed in a solution comprising phosphate, NaCl and Tween 80, wherein the phosphate has a concentration between 10 and 100 mM, the NaCl has a concentration between 100 and 500 mM, and the Tween 80 has a concentration between 0 to 0.01%, optionally wherein the solution comprises 20 mM phosphate pH 7.0, 150 mM NaCl and 001% Tween 80. In some embodiments, the antibody is digested prior to hydrophobic interaction chromatography.

In one embodiment of any one of the methods provided, the antibody isotype is IgE, IgG, IgA, IgM, or IgD. In some embodiments, the antibody isotype is IgG. In some embodiments, the antibody is Herceptin.

In some embodiments, the digestion is performed by an enzyme. In some embodiments, the enzyme is a cysteine protease. In some embodiments, the cysteine protease is papain. In some embodiments, the papain is immobilized on a solid support.

In another aspect, methods include subjecting an Fc fragment sequentially to (a) hydrophobic interaction chromatography; and (b) ultrafiltration, wherein the ultrafiltration is performed in a solution comprising phosphate, NaCl and Tween 80, wherein the phosphate has a concentration between 10 and 100 mM, the NaCl has a concentration between 100 and 500 mM, and the Tween 80 has a concentration between 0 to 0.01%, optionally wherein the solution comprises 20 mM phosphate pH 7.0, 150 mM NaCl and 001% Tween 80.

In some embodiments, the purity of the isolated Fc fragment is at least 95%, 96%, 97%, 98%, 99%, 99.5% or 99.9%. In some embodiments, the purity of the isolated Fc fragment is assessed by high performance liquid chromatography, SDS-PAGE gel electrophoresis, or contaminant protein ELISA.

In some embodiments, the hydrophobic interaction chromatography is performed using a hydrophobic chromatography column comprising an organic polymer resin. In some embodiments, the organic polymer resin is phenyl organic polymer resin.

In some embodiments, the hydrophobic interaction column is eluted using a salt buffer. In some embodiments, the elution of the hydrophobic interaction column is performed using a decreasing gradient of the salt buffer concentration. In some embodiments, ultrafiltration is performed using gel filtration chromatography.

In one embodiment of any one of the methods provided, the Fc fragment has anti-inflammatory properties. In another embodiment of any one of the methods provided, the Fc fragment is used to treat a subject with an autoimmune condition or an inflammatory condition.

In another aspect, a purified Fc fragment is produced by the method of any one of the methods provided herein.

In yet another aspect, methods involve administering a therapeutically effective amount of an Fc fragment produced in a transgenic non-human mammal to a subject in need thereof. In some embodiments, the subject has an inflammatory condition or an autoimmune condition.

Further aspects of the invention relate to a transgenic Fc fragment. In some embodiments, the transgenic Fc fragment is purified.

Further aspects of the invention relate to methods comprising administering a therapeutically effective amount of a transgenic Fc fragment to a subject in need thereof. In some embodiments, the transgenic Fc fragment is purified. In some embodiments, the subject has an inflammatory condition or an autoimmune condition.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The accompanying drawings are not intended to be drawn to scale. The Figures are illustrative only and are not required for enablement of the disclosure. For purposes of clarity, not every component may be labeled in every drawing. In the drawings:

FIG. 1 shows a representative stained SDS-PAGE protein gel with the indicated samples following Sepharose clarification.

FIG. 2 shows a representative stained SDS-PAGE protein gel with transgenically produced Herceptin/trastuzumab following digestion with papain, pepsin, ficin or trypsin.

FIG. 3 shows a non-limiting example of a work flow for digestion of transgenically-produced Herceptin/trastuzumab, followed by purification of resulting Fc fragments.

FIG. 4A shows a non-limiting example of a trace from a preparation of transgenically-produced Herceptin/trastuzumab that has been digested with papain then applied to a XK16/30 Tosoh Phenyl 650C hydrophobic interaction chromatography column. The lower trace depicts the UV absorbance at wavelength 280 nm. The middle trace depicts the pH, and the upper trace shows the conductivity. The Phenyl column was equilibrated in 20 mM phosphate pH 7.0 with 1 M sodium sulfate. FIG. 4B shows a stained SDS-PAGE protein gel with samples from the digestion with papain (Pap digest) and samples recovered from the hydrophobic interaction chromatography column including flow through (FT) and peaks 1, 2, 3, and 4 (pk 1, 2, 3, 4) under reducing (“Red”) or non-reducing (“NonRed”) conditions.

FIG. 5A shows a non-limiting example of a trace from a preparation of transgenically-produced Herceptin/trastuzumab that has been digested with papain, applied to a hydrophobic interaction chromatography column to separate the Fc and Fab fragments, then applied to a XK16/95 Superdex 200 column. The lower trace depicts the UV absorbance at wavelength 280 nm. The middle trace depicts the pH, and the upper trace shows the conductivity. The column was eluted with an isocratic run using 20 mM phosphate pH 7.0 and 150 mM NaCl. FIG. 5B shows a stained SDS-PAGE protein gel of samples recovered from the column in FIG. 5A.

FIG. 6A shows a non-limiting example of a trace from a preparation of transgenically-produced Herceptin/trastuzumab that has been digested with papain, and then applied to a XK16/30 Tosoh Phenyl 650C hydrophobic interaction chromatography column. The lower trace depicts the UV absorbance at wavelength 280 nm. The middle trace depicts the pH, and the upper trace shows the conductivity. The Phenyl column was equilibrated in 20 mM phosphate pH 7.0 with 1 M sodium sulfate. FIG. 6B shows a stained protein gel, including samples from the digestion with papain (Pap digest) and samples recovered from the hydrophobic interaction chromatography column of FIG. 6A, including flow through (FT) and peaks 1, 2, and 3 (pk 1, 2, 3) under reducing (“Red”) or non-reducing (“NonRed”) conditions.

FIG. 7 shows a non-limiting example of a trace from a preparation of transgenically-produced Herceptin/trastuzumab that was digested with papain, applied to a hydrophobic interaction chromatography column, and then applied to a XK26/95 Superdex 200 column. The lower trace depicts the UV absorbance at wavelength 280 nm. The middle trace depicts the pH, and the upper trace shows the conductivity. The column was eluted with an isocratic run using 20 mM phosphate pH 7.0 and 150 mM NaCl.

FIG. 8 shows a representative stained SDS-PAGE protein gel with samples of transgenically produced Herceptin/trastuzumab that had been applied to a hydrophobic interaction chromatography column, then subjected to gel filtration chromatography on a XK26/95 Superdex 200 column.

FIGS. 9A-D show representative traces from HPLC-SEC analysis of Fc fragments isolated from Herceptin/trastuzumab.

FIG. 10 shows a representative trace from HPLC-SEC analysis of Fc fragments isolated from Herceptin/trastuzumab.

### DETAILED DESCRIPTION OF THE INVENTION

Disclosed herein are methods, cells and transgenic animals for the production of antibodies containing an Fc fragment, and methods of isolating and purifying the Fc fragment from such antibodies. Also disclosed herein are methods, cells and transgenic animals for the production, isolation, and purification of Fc fragments. Production of many therapeutic molecules relies on recombinant expression in cells or organisms and/or extraction from natural sources. For example, Intravenous immunoglobulin (IVIG) contains anti-inflammatory IgG antibodies and is extracted from human serum. The anti-inflammatory properties of the heterogeneous IVIG are attributed to the Fc fragment of the antibodies (Samuelsson et al., (2001) Science). Described herein are alternative methods for the production of Fc fragments. These methods result in a surprisingly high level of purity of the isolated Fc fragment and overcome difficulties of traditional purification methods for isolating Fc fragments.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### Fc Fragments

Aspects of the invention relate to methods of producing a fraction crystallizable (Fc) fragment. As used herein, an “Fc fragment” refers to the portion of an immunoglobulin that interacts with cell surface Fc receptors. An Fc fragment comprises two polypeptide fragments and may be covalently linked by one or more disulfides. Each of the two polypeptide fragments may comprise one or more heavy chain constant domains selected from CH2, CH3, and CH4. In some embodiments, the Fc fragment comprises heavy chain constant domains CH2 and CH3. Fc fragments from immunoglobulins of any isotype (e.g., IgG, IgA, IgD, IgE, IgM) can be compatible with aspects of the invention. In some embodiments, the Fc fragment is an IgG Fc fragment. In some embodiments, the Fc fragment comprises the sequence provided by SEQ ID NO: 1. In some embodiments, the Fc fragment is a hybrid Fc fragment, such as is disclosed in and incorporated by reference from U.S. Pat. No. 7,867,491.

The amino acid sequence of the Fc fragment of trastuzumab is provided in SEQ ID NO:1:

APELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNV
FSCSVMHEALHNHYTQKSLSLSPGK

An Fc fragment associated with the invention may comprise one or more N-glycans at the Fc-gamma glycosylation site in the heavy chain (Asn297) of the Fc fragment. A variety of glycosylation patterns can occur at the Fc gamma glycosylation site. Oligosaccharides found at this site include galactose, N-acetylglucosamine (GlcNac), mannose, sialic acid, N-acetylneuraminic acid (NeuAc or NANA), N-glycolylneuraminic (NGNA) and fucose. N-glycans found at the Fc gamma glycosylation site generally have a common core structure consisting of an unbranched chain of a first N-acetylglucosamine (GlcNAc), which is attached to the asparagine of the antibody, a second GlcNAc that is attached to the first GlcNac and a first mannose that is attached to the second GlcNac. Two additional mannoses are attached to the first mannose of the GlcNAc-GlcNAc-mannose chain to complete the core structure, providing two “arms” for additional glycosylation. In addition, fucose residues can be attached to the N-linked first GlcNAc.

Aspects of the invention relate to isolation of Fc fragments from antibodies comprising Fc fragments. Any antibody comprising an Fc fragment can be compatible with aspects of the invention. Some aspects relate to isolation of Fc fragments that are not derived from antibodies. Fc fragments can be native Fc fragments, meaning the Fc fragment comprises the native or natural amino acid sequence of the region of the antibody from which the Fc fragment is isolated. In some embodiments, a native Fc fragment is not further modified, prior to or after isolation of the Fc fragment relative to the antibody from which the Fc fragment is isolated. In other embodiments, Fc fragments can be variant Fc fragments. Variant Fc fragments include Fc fragments which contain an amino acid sequence that differs from the native or natural amino acid sequence of the region of the antibody from which the Fc fragment is isolated. For example, the amino acid sequence can be mutated (e.g., through one or more substitutions, insertions, and/or deletions of amino acid residues). A variant Fc fragment may have been modified prior to or after isolation of the Fc fragment from the antibody. In some embodiments, the variant Fc fragments comprises additional glycosylation moieties compared to a native Fc fragment.

In some embodiments, a native Fc fragment is an IgG, IgA, IgD, IgE or IgM native Fc fragment. In some embodiments, a variant Fc fragment is an IgG, IgA, IgD, IgE or IgM variant Fc fragment.

As used herein, the term “antibody” refers to a polypeptide comprising at least two heavy (H) chains and two light (L) chains. The terms “antibody” and “immunoglobulin” are used interchangeably herein and are equivalent. Each heavy chain of an antibody is comprised of a heavy chain variable region (abbreviated herein as HCVR or VH) and a heavy chain constant region. The heavy chain constant region is comprised of at least three domains, CH1, CH2, CH3, and optionally CH4. Each light chain of an antibody is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions within the Fc fragment of the antibodies may mediate the binding of the immunoglobulin to host tissues or factors, including various cells of the immune system (e.g., effector cells) and the first component (C1q) of the classical complement system.

In some embodiments the antibodies are of the isotype IgG, IgA or IgD. In further embodiments, the antibodies are selected from the group consisting of IgG1, IgG2, IgG3, IgG4, IgM, IgA1, IgA2, IgAsec, IgD and IgE or have immunoglobulin constant and/or variable domains of IgG1, IgG2, IgG3, IgG4, IgM, IgA1, IgA2, IgAsec, IgD or IgE. In other embodiments, the antibodies are bispecific or multispecific antibodies. According to an alternative embodiment, the antibodies of the present disclosure can be modified to be in the form of a bispecific antibody, or a multispecific antibody. The term “bispecific antibody” is intended to include any agent, e.g., a protein, peptide, or protein or peptide complex, which has two different binding specificities which bind to, or interact with (a) a cell surface antigen and (b) an Fc receptor on the surface of an effector cell. The term “multispecific antibody” is intended to include any agent, e.g., a protein, peptide, or protein or peptide complex, which has more than two different binding specificities which bind to, or interact with (a) a cell surface antigen, (b) an Fc receptor on the surface of an effector cell, and (c) at least one other component. Accordingly, the disclosure includes, but is not limited to, bispecific, trispecific, tetraspecific, and other multispecific antibodies which are directed to cell surface antigens, and to Fc receptors on effector cells.

In other embodiments, the antibodies are heavy chain antibodies. The term “heavy chain antibody” refers to a polypeptide that has two heavy chains and no light chains. Each of the heavy chains of the heavy chain antibody is comprised of a heavy chain constant (CH) region and a heavy chain variable (VH) region. In some embodiments, the heavy chain constant is comprised of at least two domains. In some embodiments, the heavy chain constant region is comprised of CH2 and CH3 domains.

The term “antibodies” also encompasses different types of antibodies, e.g., recombinant antibodies, monoclonal antibodies, humanized antibodies or chimeric antibodies, or a mixture of these.

In some embodiments, the antibodies are recombinant antibodies. The term “recombinant antibody”, as used herein, is intended to include antibodies that are prepared, expressed, created or isolated by recombinant means, such as antibodies isolated from an animal that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

In yet other embodiments, the antibodies can be chimeric or humanized antibodies. As used herein, the term “chimeric antibody” refers to an antibody that combines parts of a non-human (e.g., mouse, rat, rabbit) antibody with parts of a human antibody. As used herein, the term “humanized antibody” refers to an antibody that retains only the antigen-binding CDRs from the parent antibody in association with human framework regions (see, Waldmann, 1991, Science 252:1657). Such chimeric or humanized antibodies retaining binding specificity of the murine antibody are expected to have reduced immunogenicity when administered in vivo for diagnostic, prophylactic or therapeutic applications according to the disclosure.

In certain embodiments, the antibodies are human antibodies. The term “human antibody,” as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo). Human antibodies are generated using transgenic mice carrying parts of the human immune system rather than the mouse system. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals results in the production of fully human antibodies to the antigen of interest. Following immunization of these mice (e.g., XenoMouse (Abgenix), HuMAb mice (Medarex/GenPharm)), monoclonal antibodies are prepared according to standard hybridoma technology. These monoclonal antibodies have human immunoglobulin amino acid sequences and therefore will not provoke human anti-mouse antibody (HAMA) responses when administered to humans. The human antibodies, like any of the antibodies provided herein can be monoclonal antibodies.

In some embodiments, the antibody is a full-length antibody. In some embodiments the full-length antibody comprises a heavy chain and a light chain. In some embodiments, the antibody is an anti-HER2 antibody. In some embodiments, the heavy chain comprises SEQ ID NO:2 and the light chain comprises SEQ ID NO:3. In some embodiments, the antibody includes an Fc portion comprising SEQ ID NO: 1. In some embodiments, the antibody is trastuzumab.

In some embodiments, the antibody consists of the heavy chain sequence of SEQ ID NO:2 and the light chain sequence of SEQ ID NO:3. In certain embodiments, the Fc fragment of the antibody consists of the sequence of SEQ ID NO:1. In some embodiments, the Fc fragment is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:1. It should be appreciated that any antibody could be compatible with aspects of the invention.

The heavy chain of trastuzumab is provided in SEQ ID NO:2:

MEFGLSWLFLVAILKGVQCEVQLVESGGGLVQPGGSLRLSCAASGFNI
KDTYIHWVRQAPGKGLEWVARIYPTNGYTRYADSVKGRFTISADTSKN
TAYLQMNSLRAEDTAVYYCSRWGGDGFYAMDYWGQGTLVTVSSASTKG
PSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKL
TVDKSRWQQGNVFSCSVMHEALHNHYTQKSLSLSPGK

The light chain of trastuzumab is provided in SEQ ID NO:3:

MDMRVPAQLLGLLLLWLRGARCDIQMTQSPSSLSASVGDRVTITCRAS
QDVNTAVAWYQQKPGKAPKLLIYSASFLYSGVPSRFSGSRSGTDFTLT
ISSLQPEDFATYYCQQHYTTPPTFGQGTKVEIKRTVAAPSVFIFPPSD
EQLKSGTASVVCLLNNFYPREAKVQWKVDNALQSGNSQESVTEQDSKD
STYSLSSTLTLSKADYEKHKVYACEVTHQGLSSPVTKSFNRGEC

Purification of Antibodies or Fc Fragments from Transgenic Animals

In one aspect, antibodies are purified from transgenic non-human mammals. In some embodiments, the antibodies are secreted into the milk of the transgenic non-human mammals. The antibodies can be purified from the milk of transgenic non-human mammals such that the antibodies are substantially pure. In some embodiments, substantially pure includes substantially free of contaminants. Such purification can result in an intermediate product that is further processed.

In one aspect, antibodies comprising Fc fragments are purified from a mammary epithelial cell that has been modified to express an antibody comprising an Fc fragment. The antibodies can be purified from a mammary epithelial cell such that the antibodies are substantially pure.

In one aspect, Fc fragments are purified from a mammary epithelial cell that has been modified to express an Fc fragment. The Fc fragments can be purified from a mammary epithelial cell such that the Fc fragments are substantially pure.

In some embodiments, substantially pure includes substantially free of contaminants. Such purification can result in an intermediate product that is further processed.

Antibodies comprising Fc fragments that are harvested from the milk of a transgenic non-human mammal or from a mammary epithelial cell can be purified using any suitable means known in the art to generate an intermediate product. Similarly, Fc fragments that are harvested from the milk of a transgenic non-human mammal or from a mammary epithelial cell can be purified using any suitable means known in the art to generate an intermediate product. In some embodiments, the antibody or Fc fragment is purified using a cream separator. Cream separators and use thereof are well known in the art. In some embodiments, the antibody or Fc fragment is purified using column chromatography. Column chromatography is well known in the art (see, e.g., Current Protocols in Essential Laboratory Techniques Unit 6.2 (2008) for general chromatography methods). In some embodiments, the antibody or Fc fragment is purified using a cream separator followed by column chromatography. In some embodiments, an antibody or an Fc fragment is purified using protein-G and/or protein-A affinity chromatography (see, e.g., Carter (2011) Exp Cell Res 317:1261-1269). In some embodiments, the antibodies or the Fc fragments are purified by immunoprecipitation (see, e.g., Current Protocols in Cell Biology Unit 7.2 (2001)).

In some aspects, antibodies comprising Fc fragments or Fc fragments are exposed to a detergent during purification. Use of detergents in polypeptide purification methods are well known in the art and may aid in dissolving cell membranes, solubilizing polypeptides, maintaining polypeptides in solution, and/or denaturing polypeptides. Non-limiting examples of detergents include, without limitation, sodium dodecyl sulfate (SDS), triton X-100, 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), NP-40, Tween 20, Tween 80, octyl glucoside, and octyl thioglucoside.

### Isolation and Purification of Fc Fragments

i. Hydrophobic Interaction Chromatography

Aspects of the invention provide methods of isolating an Fc fragment from an antibody by subjecting the antibody to hydrophobic interaction chromatography, followed by further purification such as by ultrafiltration. Other aspects of the invention provide methods of isolating an Fc fragment by subjecting the Fc fragment to hydrophobic interaction chromatography, followed by further purification such as by ultrafiltration. As used herein, “hydrophobic interaction chromatography” (HIC) refers to a method of separating components (e.g., antibodies, Fc fragment) in a mixture based on reversible interactions between the components and an immobilized ligand within a column, wherein hydrophobic amino acid residues within a polypeptide interact with a hydrophobic ligand contained within the column. Components can be eluted from the column by altering concentrations of a buffer, such as by applying a decreasing concentration gradient of a salt buffer. Molecules within a mixture can have different hydrophobicity characteristics, so interaction with the ligand of the column can be disrupted at different concentrations of the buffer. In some embodiments, samples from the elution of the column are collected at each concentration of the buffer for further analysis and/or purification. In some embodiments, only a sample of the elution that comprises or is suspected to comprise the Fc fragment is collected for further analysis and/or purification.

As used herein, “resin” refers to a matrix, such as a matrix comprising beads attached to a ligand. Selection of an appropriate resin will be familiar to one of ordinary skill in the art and depends on characteristics of the components of the mixture, which will be applied to the resin. In some embodiments, the resin used for an HIC column comprises an organic polymer ligand. Non-limiting examples of organic polymer resins include phenyl, ether, butyl, hexyl, or polypropylene glycol resin. In some embodiments the organic polymer resin is phenyl organic polymer resin (e.g. Tosoh Phenyl 650C). It should be appreciated that a variety of resins can be compatible with aspects of the invention. For example, the resin can be a resin produced by Tosoh Bioscience LLC, King of Prussia, Pa. In some embodiments the resin is Tosoh phenyl 650C, Tosoh phenyl 600M, Tosoh butyl 600M or Tosoh PPG 600M.

ii. Antibody Digestion

In some embodiments of the invention, an antibody is digested prior to subjecting the antibody to HIC. The antibody can be digested by any method known in the art, including, without limitation, enzymatic, chemical, or mechanical digestion methods. In some embodiments, the digestion of the antibody is performed by an enzyme. Non-limiting examples of enzymes for use in digesting antibodies include cysteine proteases such as papain and ficin, aspartate proteases such as pepsin, and serine proteases such as trypsin. In preferred embodiments, the antibody is digested at a site between the Fc fragment and the additional fragment of the antibody. In preferred embodiments, the enzyme does not digest the Fc fragment but separates the Fc fragment from the additional fragment of the antibody. In some embodiments, the digestion results in production of an Fc fragment and an additional fragment. In some embodiments, the additional fragment is a Fab fragment, a Fab′ fragment, a F(ab′)2 fragment or a scFv fragment.

Digestion may be performed at a temperature in which the enzyme is active. An appropriate duration of the digestion will be evident to one of ordinary skill in the art and can be determined using routine methods known in the art. An enzyme used to digest an antibody can be provided in a form that is immobilized on a solid support, or in a free form, or in any other form that is compatible with methods described herein. As used herein, “immobilized on a solid support” refers to a ligand (e.g., polymer, enzyme) that is attached to a resin, for example agarose beads.

iii. Ultrafiltration and Gel Filtration

Aspects of the invention relate to subjecting an antibody containing an Fc fragment to HIC, followed by further purification, such as ultrafiltration. Other aspects of the invention relate to subjecting an Fc fragment to HIC, followed by further purification, such as ultrafiltration. In some embodiments, the HIC elution samples that comprise or are suspected to comprise the Fc fragment are subjected to ultrafiltration. As used herein, “ultrafiltration” refers to a method of separating components of a mixture based on the size or molecular weight of the components. Ultrafiltration can involve in some embodiments a permeable membrane filter through which molecules smaller than the pores of the membrane are allowed to pass through whereas larger molecules are excluded and retained on the membrane. Alternatively, gel filtration chromatography, also called size exclusion chromatography, can be used to separate components of a mixture. As used herein, “gel filtration chromatography” refers to a method for separating components of a mixture based on the size or molecular weight of the components, and based on the interaction between the components of the mixture and resin within a column. Selection of a resin compatible with aspects of the invention will be familiar to one of skill in the art. In some embodiments, a polymer resin is used for gel filtration chromatography. In some embodiments, the polymer resin is a dextran resin (e.g. Superdex 200™).

The antibodies, fragments thereof, or Fc fragments can be eluted from the column by applying a buffer at various concentrations. In some embodiments samples from the elution are collected at each concentration of the buffer for further analysis. In some embodiments, only a sample of the elution that comprises or is suspected to comprise the Fc fragment is collected for further analysis.

The elution samples comprising the antibody, fragments thereof, or Fc fragment can be further analyzed or assessed for purity by any method known in the art including, without limitation, Western blotting, protein electrophoresis, protein staining, high performance liquid chromatography or mass spectrometry.

iv. Buffer Conditions

It should be appreciated that a variety of solutions, such as buffers, can be compatible with aspects of the invention. In some embodiments, digestion of an antibody comprising an Fc fragment occurs in a tromethamine (tris) buffer. In some embodiment the buffer is a tris-phosphate buffer. In other embodiments the buffer is a phosphate buffer. In some embodiments, the buffer has a concentration of between 1 mM and 100 mM, between 2 mM and 50 mM, or between 5 mM and 20 mM. In some embodiments, the buffer concentration is less than 1 mM. In some embodiments, the buffer concentration is more than 100 mM. In some embodiments, the buffer concentration is approximately 20 mM. It should be appreciated that the buffer concentration is dependent on the nature of the buffer that is being used.

In some embodiments, the pH of the buffer is between pH 6 and pH 9 or between pH 6.5 and pH 7.5. For example, in some embodiments, the pH is approximately 6.5, 6.6, 6.7, 6.8, 6.9, 7.0, 7.1, 7.2, 7.3, 7.4 or 7.5. In some embodiments, the pH of the buffer is approximately 7.0. If needed, acid (such as HCL) or base (such as NaOH) can be added to the buffer to attain the desired pH. Ethylenediaminetetraacetic acid (EDTA) may be added to the buffer to chelate multivalent cations. In some embodiments, the EDTA concentration is between 1 mM and 100 mM, between 2 mM and 50 mM, or between 5 mM and 20 mM. In some embodiments, the EDTA concentration is approximately 10 mM.

In some embodiments, the HIC column is eluted using a salt buffer or concentration gradient thereof. In some embodiments, the salt buffer is a sodium sulfate buffer. In some embodiments, the concentration range of a salt buffer used to elute a HIC column can be from 0.1M to 1M, or between 0.3 M and 0.8M, or between 0.4M to 0.6M.

In some embodiments, a buffer used for eluting an Fc fragment following gel filtration chromatography is a tromethamine (tris) buffer. In some embodiments, the buffer is a tris-phosphate buffer. In other embodiments the buffer is a phosphate buffer. In some embodiments, the buffer is present in a concentration of between 1 mM and 100 mM, between 2 mM and 50 mM, or between 5 mM and 20 mM. In some embodiments, the buffer concentration is less than 1 mM. In some embodiments, the buffer concentration is more than 100 mM. In some embodiments, the buffer concentration is approximately 20 mM. In some embodiments, the buffer is a phosphate buffer at a concentration of approximately 20 mM. In some embodiments, the pH of the buffer is between pH 6 and pH 9 or between pH 6.5 and pH 7.5. For example, in some embodiments, the pH is approximately 6.5, 6.6, 6.7, 6.8, 6.9, 7.0, 7.1, 7.2, 7.3, 7.4 or 7.5. In some embodiments, the pH of the buffer is approximately 7.0. In some embodiments, the buffer may be further supplemented with a salt, such as potassium chloride or sodium chloride. In some embodiments, the salt concentration in the buffer is between 1 mM and 300 mM, between 50 mM and 200 mM, or between 100 mM and 200 mM. In some embodiments, the salt concentration is approximately 150 mM. In some embodiments, sodium chloride is added to the buffer at a concentration of 150 mM.

In some embodiments, buffers further comprise a detergent, such as Polysorbate 80 (Tween 80). In some embodiments, the concentration of detergent in the buffer is between 0.001% and 1%, or between 0.05% and 0.1%. In some embodiments, the concentration of detergent, such as Tween 80 in the buffer is approximately 0.01%.

### Constructs for the Generation of Transgenic Animals Expressing Antibodies

Some aspects of the invention relate to producing primary cell lines containing a construct (e.g., encoding an Fc fragment or an antibody comprising an Fc fragment) for use in producing transgenic goats by nuclear transfer. The constructs can be transfected into primary goat skin epithelial cells, which are clonally expanded and fully characterized to assess transgene copy number, transgene structural integrity and chromosomal integration site. As used herein, “nuclear transfer” refers to a method of cloning wherein the nucleus from a donor cell is transplanted into an enucleated oocyte.

Coding sequences for proteins of interest (e.g., an Fc fragment or an antibody comprising an Fc fragment) can be obtained from any suitable source including by screening libraries of genomic material or reverse-translated messenger RNA derived from the animal of choice (such as an equine), obtained from sequence databases such as NCBI, Genbank, or by obtaining the sequences of the antibody or Fc fragment, etc. The sequences can be cloned into an appropriate plasmid vector and amplified in a suitable host organism, like E. coli. After amplification of the vector, the DNA construct can be excised, purified from the remains of the vector and introduced into expression vectors that can be used to produce transgenic animals. The transgenic animals will have the desired transgenic protein integrated into their genome.

After amplification of the vector, the DNA construct can also be excised with the appropriate 5′ and 3′ control sequences, purified away from the remains of the vector and used to produce transgenic animals that have integrated into their genome the desired expression constructs. Conversely, with some vectors, such as yeast artificial chromosomes (YACs), it is not necessary to remove the assembled construct from the vector; in such cases the amplified vector may be used directly to make transgenic animals. The coding sequence can be operatively linked to a control sequence, which enables the coding sequence to be expressed in the milk of a transgenic non-human mammal.

A DNA sequence which is suitable for directing production of an Fc fragment or an antibody comprising an Fc fragment, to the milk of transgenic animals can carry a 5′-promoter region derived from a naturally-derived milk protein. This promoter is consequently under the control of hormonal and tissue-specific factors and is most active in lactating mammary tissue. In some embodiments, the promoter is a caprine beta casein promoter. The promoter can be operably linked to a DNA sequence directing the production of a protein leader sequence, which directs the secretion of the transgenic protein across the mammary epithelium into the milk. In some embodiments, a 3′-sequence, which can be derived from a naturally secreted milk protein, can be added to improve stability of mRNA. As used herein, a “leader sequence” or “signal sequence” is a nucleic acid sequence that encodes a protein secretory signal, and, when operably linked to a downstream nucleic acid molecule encoding a transgenic protein directs secretion. The leader sequence may be the native human leader sequence, an artificially-derived leader, or may obtained from the same gene as the promoter used to direct transcription of the transgene coding sequence, or from another protein that is normally secreted from a cell, such as a mammalian mammary epithelial cell.

In some embodiments, the promoters are milk-specific promoters. As used herein, a “milk-specific promoter” is a promoter that naturally directs expression of a gene in a cell that secretes a protein into milk (e.g., a mammary epithelial cell) and includes, for example, the casein promoters, e.g., α-casein promoter (e.g., alpha S-1 casein promoter and alpha S2-casein promoter), β-casein promoter (e.g., the goat beta casein gene promoter (DiTullio, BIOTECHNOLOGY 10:74-77, 1992), γ-casein promoter, κ-casein promoter, whey acidic protein (WAP) promoter (Gordon et al., BIOTECHNOLOGY 5: 1183-1187, 1987), β-lactoglobulin promoter (Clark et al., BIOTECHNOLOGY 7: 487-492, 1989) and α-lactalbumin promoter (Soulier et al., FEBS LETTS. 297:13, 1992). Also included in this definition are promoters that are specifically activated in mammary tissue, such as, for example, the long terminal repeat (LTR) promoter of the mouse mammary tumor virus (MMTV).

As used herein, a coding sequence and regulatory sequence are said to be “operably joined” when they are covalently linked in such a way as to place the expression or transcription of the coding sequence under the influence or control of the regulatory sequences. In order for the coding sequences to be translated into a functional protein the coding sequences are operably joined to regulatory sequences. Two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region were capable of effecting transcription of that DNA sequence such that the resulting transcript might be translated into the desired polypeptide (e.g., Fc fragment or antibody).

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium, or just a single time per host as the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells, which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques. Preferred vectors are those capable of autonomous replication and expression of the structural gene products present in the DNA segments to which they are operably joined.

### Mammary Epithelial Cells and Transgenic Animals for Production of Fc Fragments

In one aspect, the disclosure provides mammary gland epithelial cells that express an antibody comprising an Fc fragment. In another aspect, the disclosure provides mammary gland epithelial cells that express an Fc fragment. In some embodiments, the disclosure provides a transgenic non-human mammal comprising mammary gland epithelial cells that express the antibody comprising an Fc fragment. In other embodiments, the disclosure provides a transgenic non-human mammal comprising mammary gland epithelial cells that express the Fc fragment.

In one aspect, the disclosure provides a method for the production of an Fc fragment or an antibody comprising an Fc fragment, comprising:
(a) transfecting non-human mammalian cells with a transgene DNA construct encoding an Fc fragment or an antibody comprising an Fc fragment;
(b) selecting cells in which said transgene DNA construct has been inserted into the genome of the cells; and
(c) performing a first nuclear transfer procedure to generate a non-human transgenic mammal heterozygous for the Fc fragment or the antibody comprising an Fc fragment, and that can express the Fc fragment or the antibody comprising an Fc fragment in its milk.
   In one aspect, the disclosure provides a method of
(a) providing a non-human transgenic mammal engineered to express an Fc fragment or an antibody comprising an Fc fragment,
(b) expressing the Fc fragment or the antibody comprising the Fc fragment in the milk of the non-human transgenic mammal; and
(c) isolating the Fc fragment or the antibody comprising the Fc fragment produced in the milk.

Transgenic animals can also be generated according to methods known in the art (See e.g., U.S. Pat. No. 5,945,577). Animals suitable for transgenic expression, include, but are not limited to goat, sheep, bison, camel, cow, rabbit, buffalo, horse, rat, mouse or llama. Suitable animals also include bovine, caprine, and ovine, which relate to various species of cows, goats, and sheep, respectively. Suitable animals also include ungulates. As used herein, “ungulate” is of or relating to a hoofed typically herbivorous quadruped mammal, including, without limitation, sheep, goats, cattle and horses. In one embodiment, the animals are generated by co-transfecting primary cells with separate constructs. These cells are then used for nuclear transfer. Alternatively, if micro-injection is used to generate the transgenic animals, the constructs may be injected.

Cloning will result in a multiplicity of transgenic animals—each capable of producing an Fc fragment or an antibody comprising an Fc fragment or other gene construct of interest. The production methods include the use of the cloned animals and the offspring of those animals. In some embodiments, the cloned animals are caprines, bovines or mice. Cloning also encompasses the nuclear transfer of fetuses, nuclear transfer, tissue and organ transplantation and the creation of chimeric offspring.

One step of the cloning process comprises transferring the genome of a cell that contains the transgene encoding the Fc fragment construct or the antibody construct into an enucleated oocyte. As used herein, “transgene” refers to any piece of a nucleic acid molecule that is inserted by artifice into a cell, or an ancestor thereof, and becomes part of the genome of an animal which develops from that cell. Such a transgene may include a gene which is partly or entirely exogenous (i.e., foreign) to the transgenic animal, or may represent a gene having identity to an endogenous gene of the animal.

Suitable mammalian sources for oocytes include goats, sheep, cows, rabbits, guinea pigs, mice, hamsters, rats, non-human primates, etc. Preferably, oocytes are obtained from ungulates, and most preferably goats or cattle. Methods for isolation of oocytes are well known in the art. Essentially, the process comprises isolating oocytes from the ovaries or reproductive tract of a mammal, e.g., a goat. A readily available source of ungulate oocytes is from hormonally-induced female animals. For the successful use of techniques such as genetic engineering, nuclear transfer and cloning, oocytes may preferably be matured in vivo before these cells may be used as recipient cells for nuclear transfer, and before they were fertilized by the sperm cell to develop into an embryo. Metaphase II stage oocytes, which have been matured in vivo, have been successfully used in nuclear transfer techniques. Essentially, mature metaphase II oocytes are collected surgically from either non-super ovulated or super ovulated animals several hours past the onset of estrus or past the injection of human chorionic gonadotropin (hCG) or similar hormone.

One of the tools used to predict the quantity and quality of the recombinant protein expressed in the mammary gland is through the induction of lactation (Ebert KM, 1994). Induced lactation allows for the expression and analysis of protein from the early stage of transgenic production rather than from the first natural lactation resulting from pregnancy, which is at least a year later. Induction of lactation can be done either hormonally or manually.

In one aspect the disclosure provides mammary gland epithelial cells and transgenic non-human mammals that produce an Fc fragment or an antibody comprising an Fc fragment. Mammary gland epithelial cells and transgenic non-human mammals according to aspects of the invention express nucleic acid sequences encoding the antibody. In some embodiments, the nucleic acid sequences comprise a sequence encoding the Fc fragment set forth in SEQ ID NO: 1.

### Production of Fc Fragments

Aspects of the invention relate to transgenic Fc fragments. In some embodiments, the transgenic Fc fragment is purified.

In some aspects, Fc fragments produced as described herein in transgenic non-human mammals or in mammary epithelial cells have altered characteristics compared to Fc fragments produced by other methods. For example, Fc fragments produced as described herein can exhibit altered glycosylation and/or sialylation compared to Fc fragments produced by other methods. Fc fragments produced as described herein can exhibit increased half-lives and/or stability compared to Fc fragments produced by other methods. Fc fragments produced as described herein can also exhibit enhanced anti-inflammatory properties when administered to a subject compared to Fc fragments produced by other methods.

In one aspect, the disclosure provides recombinant or transgenically produced antibodies wherein the Fc fragments are subsequently isolated and purified from the antibodies. In another aspect, the disclosure provides transgenically produced Fc fragments that are subsequently isolated and purified. Such Fc fragments and compositions comprising recombinant or transgenically produced Fc fragments can exhibit glycosylation and/or siaylation. For example, in some embodiments, Fc fragments produced in mammary epithelial cells of a non-human mammal, and then isolated and purified, have increased levels of glycosylation and or sialylation when compared to Fc fragments not produced in mammary gland epithelial cells. In some embodiments, the Fc fragments not produced in mammary gland epithelial cells are produced in cell culture. As used herein, Fc fragments “produced in cell culture” when compared to Fc fragments produced in mammary epithelial cells, refers to Fc fragments produced in standard production cell lines (e.g., CHO cells or baculovirus-Sf9 cells) but excluding mammary epithelial cells. In some embodiments, Fc fragments produced in mammary epithelial cells of a non-human mammal, and then isolated and purified, have increased levels of glycosylation and or sialylation when compared to Fc fragments isolated from IVIG.

In some embodiments the methods above further comprise steps for inducing lactation. In some embodiments the methods further comprise additional isolation and/or purification steps. In yet other embodiments the methods further comprise steps for comparing the glycosylation pattern of the Fc fragments produced in cell culture, e.g. non-mammary cell culture. In further embodiments, the methods further comprise steps for comparing the glycosylation pattern of the Fc fragments obtained to Fc fragments produced by non-mammary epithelial cells. Such cells can be cells of a cell culture. Experimental techniques for assessing the glycosylation pattern of the Fc fragments are known to those of ordinary skill in the art. Such methods include, e.g., liquid chromatography mass spectrometry, tandem mass spectrometry, and Western blot analysis.

In some aspects, the Fc fragments disclosed herein are generated by producing an antibody comprising an Fc fragment in a transgenic non-human mammal or in mammary epithelial cells. In other embodiments, the Fc fragments disclosed herein are generated by producing the Fc fragments in a transgenic non-human mammal or in mammary epithelial cells. In some embodiments, it may be advantageous to increase the sialylation level of the Fc fragments. The sialylation levels of the Fc fragments can be increased for instance by subjecting the Fc fragment or the antibody comprising the Fc fragment to sialyl transferases. The Fc fragment or the antibody comprising the Fc fragment can be subjected to sialyl transferases in vitro or in vivo. The Fc fragment or the antibody comprising the Fc fragment can be sialylated in vitro by subjecting the Fc fragment or the antibody comprising the Fc fragment to a sialyl transferase and the appropriate saccharide based substrate. The Fc fragment or the antibody comprising the Fc fragment can be sialylated in vivo by producing a sialyl transferase in the mammary gland or mammary epithelial cells.

In some aspects, the disclosure provides methods for the production in the mammary gland of transgenic animals, or in mammary epithelial cells, of an Fc fragment or an antibody comprising an Fc fragment with increased levels of alpha-2,6-sialylation. Fc fragments that exhibits increased sialylation may exhibit increased anti-inflammatory properties.

In one aspect, the disclosure provides transgenic animals (and mammary epithelial cells) that are transgenic for the production in the mammary gland of an Fc fragment or an antibody comprising an Fc fragment, and that are also transgenic for the production of sialyl transferase. The Fc fragments produced by such animals and cells are expected to have increased levels of terminal alpha-2,6-sialic acid linkages.

In one aspect, the disclosure provides methods of treating a subject comprising administering to a subject the Fc fragments that have increased levels of terminal alpha-2,6-sialic acid linkages.

The Fc fragment can be obtained, in some embodiments, by harvesting the Fc fragment or the antibody comprising the Fc fragment, from the milk of a transgenic animal produced as provided herein or from an offspring of said transgenic animal. In some embodiments the Fc fragment is produced at a level of at least 1 gram per liter of milk produced. For example, in some embodiments, methods described herein allow for production of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 grams per liter of an Fc fragment. In some embodiments the antibody is produced at a level of at least 1 gram per liter of milk produced. For example, in some embodiments, methods described herein allow for production of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 grams per liter of an antibody.

In some aspects, the Fc fragments produced as described herein have enhanced characteristics compared to Fc fragments produced by other methods. For example, in some embodiments, Fc fragments produced by methods described herein are of higher purity compared to Fc fragments produced by other methods. In some embodiments, the transgenically produced Fc fragments that are subsequently isolated and purified are at least 95-99.99% pure. In some embodiments, the transgenically produced Fc fragments that are subsequently isolated and purified are at least 95, 96, 97, 98, 99, 99.5, or 99.99% pure. In some embodiments, the Fc fragments isolated and purified from the transgenically produced antibodies are at least 95-99.99% pure. In some embodiments, the Fc fragments isolated and purified from the transgenically produced antibodies are at least 95, 96, 97, 98, 99, 99.5, or 99.99% pure.

Purity of Fc fragments produced by any of the methods described herein may be assessed by any technique known to those of skill in the art, including, without limitation, Western blotting, protein electrophoresis, protein staining, high performance liquid chromatography, mass spectrometry, contaminant protein ELISA, etc.

Fc fragments produced as described herein can also be produced with enhanced efficiency compared to Fc fragments produced by other methods. As used herein, “enhanced efficiency” refers to a higher percent yield of Fc fragments relative to the starting material. In some embodiments, the percent yield of Fc fragments isolated and purified from the transgenically produced antibodies is 60-80%. In some embodiments, the percent yield of Fc fragments isolated and purified from the transgenically produced antibodies is at least 60, 65, 70, 75, or 80%.

### Methods of Treatment

In some aspects, the disclosure provides methods of administering an Fc fragment or compositions comprising an Fc fragment to a subject in need thereof. Methods for determining whether a subject is in need of a treatment comprising an Fc fragment are known in the art. For example, in some embodiments, a subject in need of a treatment comprising administering an Fc fragment or composition comprising an Fc fragment is a subject having an autoimmune condition or an inflammatory condition. Exemplary autoimmune conditions and/or inflammatory conditions that could be treated by practice of the invention described herein will be apparent to one of skill in the art. Non-limiting examples are specifically incorporated by reference from U.S. Pat. No. 8,349,793 and PCT publication WO2013/034738.

Non-limiting examples of autoimmune conditions include Acute disseminated encephalomyelitis (ADEM), Addison's disease, Agammaglobulinemia, Alopecia areata, Amyotrophic lateral sclerosis (Also Lou Gehrig's disease; Motor Neuron Disease), Ankylosing Spondylitis, Antiphospholipid syndrome, Antisynthetase syndrome, Atopic allergy, Atopic dermatitis, Autoimmune aplastic anemia, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenic purpura, Autoimmune urticarial, Autoimmune uveitis, Balo disease/Balo concentric sclerosis, Behçet's disease, Berger's disease, Bickerstaff s encephalitis, Blau syndrome, Bullous pemphigoid, Castleman's disease, Celiac disease, Chagas disease, Chronic inflammatory demyelinating polyneuropathy, Chronic recurrent multifocal osteomyelitis, Chronic obstructive pulmonary disease, Churg-Strauss syndrome, Cicatricial pemphigoid, Cogan syndrome, Cold agglutinin disease, Complement component 2 deficiency, Contact dermatitis, Cranial arteritis, CREST syndrome, Crohn's disease, Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Drug-induced lupus, Discoid lupus erythematosus, Eczema, Endometriosis, Enthesitis-related arthritis, Eosinophilic fasciitis, Eosinophilic gastroenteritis, Eosinophilic pneumonia, Epidermolysis bullosa acquisita, Erythema nodosum, Erythroblastosis fetalis, Essential mixed cryoglobulinemia, Evan's syndrome, Fibrodysplasia ossificans progressive, Fibrosing alveolitis (or Idiopathic pulmonary fibrosis), Gastritis, Gastrointestinal pemphigoid, Glomerulonephritis, Goodpasture's syndrome, Graves' disease, Guillain-Barre syndrome (GBS), Hashimoto's encephalopathy, Hashimoto's thyroiditis, Henoch-Schonlein purpura, Herpes gestationis aka Gestational Pemphigoid, Hidradenitis suppurativa, Hughes-Stovin syndrome, Hypogammaglobulinemia, Idiopathic inflammatory demyelinating diseases, Idiopathic pulmonary fibrosis, Idiopathic thrombocytopenic purpura (See Autoimmune thrombocytopenic purpura), IgA nephropathy, Inclusion body myositis, Chronic inflammatory demyelinating polyneuropathy, Interstitial cystitis, Juvenile idiopathic arthritis aka Juvenile rheumatoid arthritis, Kawasaki's disease, Lambert-Eaton myasthenic syndrome, Leukocytoclastic vasculitis, Lichen planus, Lichen sclerosus, Linear IgA disease (LAD), Lupoid hepatitis aka Autoimmune hepatitis, Lupus erythematosus, Majeed syndrome, Meniere's disease, Microscopic polyangiitis, Miller-Fisher syndrome, Mixed connective tissue disease, Morphea, Mucha-Habermann disease aka Pityriasis lichenoides et varioliformis acuta, Multiple sclerosis, Myasthenia gravis, Myositis, Narcolepsy, Neuromyelitis optica (also Devic's disease), Neuromyotonia, Occular cicatricial pemphigoid, Opsoclonus myoclonus syndrome, Ord's thyroiditis, Palindromic rheumatism, PANDAS (pediatric autoimmune neuropsychiatric disorders associated with streptococcus), Paraneoplastic cerebellar degeneration, Paroxysmal nocturnal hemoglobinuria (PNH), Parry Romberg syndrome, Parsonage-Turner syndrome, Pars planitis, Pemphigus vulgaris, Pernicious anaemia, Perivenous encephalomyelitis, POEMS syndrome, Polyarteritis nodosa, Polymyalgia rheumatic, Polymyositis, Primary biliary cirrhosis, Primary sclerosing cholangitis, Progressive inflammatory neuropathy, Psoriasis, Psoriatic arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatic fever, Sarcoidosis, Schizophrenia, Schmidt syndrome another form of APS, Schnitzler syndrome, Scleritis, Scleroderma, Serum Sickness, Sjögren's syndrome, Spondyloarthropathy, Still's disease, Stiff person syndrome, Subacute bacterial endocarditis (SBE), Susac's syndrome, Sweet's syndrome, Sydenham chorea see PANDAS, Sympathetic ophthalmia, Systemic lupus erythematosus see Lupus erythematosus, Takayasu's arteritis, Temporal arteritis (also known as “giant cell arteritis”), Thrombocytopenia, Tolosa-Hunt syndrome, Transverse myelitis, Ulcerative colitis, Undifferentiated connective tissue disease, Undifferentiated spondyloarthropathy, Urticarial vasculitis, Vasculitis, Vitiligo and Wegener's granulomatosis.

Non-limiting examples of inflammatory conditions include Ankylosing Spondylitis (AS), Antiphospholipid Antibody Syndrome (APS), Gout, Inflammatory Arthritis Center, Myositis, Rheumatoid Arthritis, Scleroderma, Sjogren's Syndrome, Systemic Lupus Erythematosus (SLE, Lupus), Vasculitis, Appendicitis, Bursitis, Colitis, Cystitis, Dermatitis, Infective meningitis, Tonsillitis, Asthma, Pneumonia, Phlebitis, RSD/CRPS, Rhinitis, Tendonitis, Tonsillitis, Vasculitis, pruritus, skin inflammation, psoriasis, atopic dermatitis, allergic contact dermatitis, irritant contact dermatitis, and seborrhoeic dermatitis, keratinopathy, inflammatory bowel disease, ulcerative colitis, Crohn's disease, multiple sclerosis, osteoarthritis, Hashimoto's thyroidis, myasthenia gravis, diabetes type I or II, inflammatory lung injury, inflammatory liver injury, inflammatory glomerular injury, keratoconjunctivitis, an inflammatory disease of the joints, skin, or muscle, acute or chronic idiopathic inflammatory arthritis, a demyelinating disease, chronic obstructive pulmonary disease, interstitial lung disease, interstitial nephritis and chronic active hepatitis.

### Pharmaceutical Compositions, Dosage, and Administration

Aspects of the invention relate to administering effective amounts of an Fc fragment, or compositions comprising an Fc fragment. In some embodiments, methods comprise administering a therapeutically effective amount of a transgenic Fc fragment to a subject in need thereof. In some embodiments, the transgenic Fc fragment is purified. In some embodiments, the subject has an inflammatory condition or an autoimmune condition.

As used herein, a “therapeutically effective amount” or an “effective amount” refers to an amount of Fc fragment or composition comprising an Fc fragment that is effective to influence a condition. For example, in some embodiments, an effective amount could be an amount that is sufficient for reducing inflammation or autoimmunity. Determining an effective amount depends on such factors as toxicity and efficacy of the composition. These factors will differ depending on other factors such as potency, relative bioavailability, subject body weight, severity of adverse side-effects and preferred mode of administration. Toxicity may be determined using methods well known in the art. Efficacy may be determined utilizing the same guidance. Efficacy, for example, can be in some embodiments measured by quantifying the amount of an inflammatory cytokine, presence of inflammatory cells, amount of specific antibodies, or characteristics such as redness or swelling. An effective amount can be readily determined by one of ordinary skill in the art.

Dosage may be adjusted appropriately to achieve desired levels, local or systemic, depending upon the mode of administration. In the event that the response in a subject is insufficient at such doses, even higher doses (or effective higher doses by a different, more localized delivery route) may be employed to the extent that subject tolerance permits. In some embodiments, multiple doses per day can be used to achieve appropriate systemic levels of a product or composition. Appropriate systemic levels can be determined by, for example, measurement of the subject's peak or sustained plasma level of the Fc fragment. “Dose” and “dosage” are used interchangeably herein.

In some embodiments, the amount of Fc fragment or pharmaceutical composition comprising an Fc fragment administered to a subject is 50 to 500 mg/kg, 100 to 400 mg/kg, or 200 to 300 mg/kg per week. In one embodiment the amount of Fc fragment or pharmaceutical composition comprising an Fc fragment administered to a subject is 250 mg/kg per week. In some embodiments, an initial dose of 400 mg/kg is administered a subject the first week, followed by administration of 250 mg/kg to the subject in subsequent weeks. In some embodiments the administration rate is less than 10 mg/min. In some embodiments, administration of the Fc fragment or pharmaceutical composition comprising an Fc fragment to a subject occurs at least one hour prior to treatment with another therapeutic agent. In some embodiments, a pre-treatment is administered prior to administration of the Fc fragment or pharmaceutical composition comprising an Fc fragment.

In some aspects, the disclosure provides compositions, including pharmaceutical compositions, which comprise transgenically produced and purified Fc fragments and a pharmaceutically acceptable vehicle, diluent or carrier.

In some embodiments the compositions provided are employed for in vivo applications. Depending on the intended mode of administration in vivo the compositions used may be in the dosage form of solid, semi-solid or liquid such as, e.g., tablets, pills, powders, capsules, gels, ointments, liquids, suspensions, or the like. Preferably, the compositions are administered in unit dosage forms suitable for single administration of precise dosage amounts. The compositions may also include, depending on the formulation desired, pharmaceutically acceptable carriers or diluents, which are defined as aqueous-based vehicles commonly used to formulate pharmaceutical compositions for animal or human administration. The diluent is selected so as not to affect the biological activity of the Fc fragment. Examples of such diluents are distilled water, physiological saline, Ringer's solution, dextrose solution, and Hank's solution. The same diluents may be used to reconstitute a lyophilized recombinant protein of interest. In addition, the pharmaceutical composition may also include other medicinal agents, pharmaceutical agents, carriers, adjuvants, nontoxic, non-therapeutic, non-immunogenic stabilizers, etc. Effective amounts of such diluent or carrier are amounts which are effective to obtain a pharmaceutically acceptable formulation in terms of solubility of components, biological activity, etc. In some embodiments the compositions provided herein are sterile.

Administration during in vivo treatment may be by any number of routes, including oral, parenteral, intramuscular, intranasal, sublingual, intratracheal, inhalation, ocular, vaginal, and rectal. Intracapsular, intravenous, and intraperitoneal routes of administration may also be employed. The skilled artisan recognizes that the route of administration varies depending on the response desired. For example, the Fc fragments or compositions herein may be administered to a subject via oral, parenteral or topical administration. In one embodiment, the compositions herein are administered by intravenous infusion.

The compositions, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compositions in water soluble form. Additionally, suspensions of the active compositions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions. Alternatively, the active compositions may be in powder form for constitution with a suitable vehicle, e.g., sterile pyrogen-free water, before use.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

The component or components may be chemically modified so that oral delivery is efficacious. Generally, the chemical modification contemplated is the attachment of at least one molecule, where said molecule permits (a) inhibition of proteolysis; and (b) uptake into the blood stream from the stomach or intestine. Also desired is the increase in overall stability and increase in circulation time in the body. Examples of such molecules include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol molecules. For oral compositions, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the biologically active material or by release of the biologically active material beyond the stomach environment, such as in the intestine. For buccal administration, the compositions may take the form of tablets or lozenges formulated in conventional manner.

The compositions may also be formulated in rectal or vaginal compositions such as suppositories or retention enemas, e.g., containing conventional suppository bases such as cocoa butter or other glycerides.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compositions, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

Therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the disclosure contain an effective amount of an Fc fragment and optionally therapeutic agents included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compositions of the present disclosure, and with each other, in a manner such that there is no interaction which would substantially impair the desired pharmaceutical efficiency.

The therapeutic agent(s), including Fc fragments, may in some embodiments be provided in particles. Particles as used herein means nano or microparticles (or in some instances larger) which can consist in whole or in part of the therapeutic agent or can include other additional therapeutic agents. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the therapeutic agent in a solution or in a semi-solid state. The particles may be of virtually any shape.

### EQUIVALENTS

Those skilled in the art will recognize, or be able to ascertain using no more than routine experimentation, many equivalents to the specific embodiments of the invention described herein. Such equivalents are intended to be encompassed by the following claims.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Example 1: Generation of Transgenic Goats that Produce Herceptin/Trastuzumab

Transgenic goats were generated that include the nucleic acid sequence encoding the trastuzumab antibody in their genome. The goats producing trastuzumab were generated using traditional microinjection techniques (See e.g., U.S. Pat. No. 7,928,064). The cDNA encoding the heavy and light chain (SEQ ID NO:4 and SEQ ID NO:5) were ligated with the beta casein expression vector to yield constructs BC2601 HC and BC2602 LC. In these plasmids, the nucleic acid sequence encoding trastuzumab is under the control of a promoter facilitating the expression of trastuzumab in the mammary gland of the goats. The prokaryotic sequences were removed and the DNA microinjected into pre-implantation embryos of the goat. These embryos were then transferred to pseudo pregnant females. The progeny that resulted were screened for the presence of the transgenes. Those that carried both chains were identified as transgenic founders.

A nucleic acid sequence encoding the heavy chain of tratsuzumab is provided in SEQ ID NO:4:

ATGGAGTTCGGCCTGAGCTGGCTGTTCCTGGTGGCCATCCTGAAGGGC
GTGCAGTGCGAGGTGCAGCTGGTCGAGAGCGGAGGAGGACTGGTCCAG
CCTGGCGGCAGCCTGAGACTGAGCTGCGCCGCCAGCGGCTTCAACATC
AAGGACACCTACATCCACTGGGTGCGCCAGGCTCCAGGGAAAGGGCTC
GAATGGGTGGCCAGGATCTACCCCACCAACGGCTACACCAGATACGCC
GACAGCGTGAAGGGCAGGTTCACCATCAGCGCCGACACCAGCAAGAAC
ACCGCCTACCTGCAGATGAACAGCCTGAGGGCCGAGGACACCGCCGTG
TACTACTGCAGCAGATGGGGTGGGGATGGCTTCTACGCCATGGACTAC
TGGGGGCAGGGCACACTGGTCACAGTCTCCAGCGCCAGCACCAAGGGC
CCCAGCGTGTTCCCCCTGGCTCCTTCCTCTAAATCCACAAGCGGCGGC
ACCGCTGCCCTGGGCTGCCTGGTGAAGGACTACTTCCCCGAGCCCGTG
ACCGTGTCTTGGAACTCTGGCGCCCTGACCTCCGGCGTGCACACCTTC
CCCGCCGTGCTGCAGAGCAGCGGCCTGTACAGCCTGAGCAGCGTGGTG
ACCGTGCCCTCTTCCTCTCTCGGAACACAGACCTACATCTGCAACGTG
AACCACAAGCCCAGCAACACCAAGGTGGACAAGAAGGTGGAGCCCAAG
AGCTGCGACAAGACCCATACATGTCCTCCCTGTCCTGCTCCTGAGCTG
CTGGGCGGACCCTCCGTGTTCCTGTTCCCCCCCAAGCCCAAGGACACC
CTGATGATCAGCAGGACCCCCGAGGTGACCTGCGTGGTGGTGGACGTG
TCCCACGAGGACCCTGAGGTGAAGTTCAACTGGTACGTGGACGGCGTG
GAGGTGCACAACGCCAAGACCAAGCCCAGAGAGGAGCAGTACAACAGC
ACCTACAGGGTGGTGTCCGTGCTGACCGTGCTGCACCAGGACTGGCTG
AACGGCAAAGAATACAAGTGCAAAGTCTCCAACAAGGCCCTGCCAGCC
CCCATCGAAAAGACCATCAGCAAGGCCAAGGGCCAGCCTCGCGAGCCC
CAGGTGTACACCCTGCCCCCCTCCCGCGACGAGCTGACCAAGAACCAG
GTGTCCCTGACCTGTCTGGTGAAGGGCTTCTACCCCAGCGATATCGCC
GTGGAGTGGGAGAGCAACGGCCAGCCCGAGAACAACTACAAGACCACC
CCCCCTGTGCTGGACAGCGACGGCAGCTTCTTCCTGTACAGCAAGCTG
ACCGTGGACAAGAGCAGGTGGCAGCAGGGAAATGTCTTTTCCTGTTCC
GTCATGCATGAAGCTCTGCACAACCACTACACCCAGAAGTCCCTGAGC
CTGAGCCCCGGCAAGTGATAG

A nucleic acid sequence encoding the light chain of tratsuzumab is provided in SEQ ID NO:5:

ATGGACATGAGAGTGCCTGCCCAGCTCCTGGGACTCCTCCTCCTGTGG
CTCAGGGGTGCTCGCTGCGATATCCAGATGACTCAGTCTCCTTCTTCC
CTCTCCGCCAGCGTGGGCGACAGAGTGACCATCACCTGCAGGGCCAGC
CAGGACGTGAACACCGCCGTGGCCTGGTATCAGCAGAAGCCCGGCAAG
GCCCCCAAGCTGCTGATCTACAGCGCCAGCTTCCTGTACAGCGGCGTG
CCCAGCAGGTTCAGCGGCAGCAGAAGCGGCACCGACTTCACCCTGACC
ATCAGCAGCCTGCAGCCCGAGGACTTCGCCACCTACTACTGCCAGCAG
CACTACACCACCCCCCCCACCTTCGGCCAGGGCACCAAGGTGGAGATC
AAGAGGACCGTGGCCGCTCCCAGCGTGTTCATCTTCCCCCCCAGCGAC
GAGCAGCTGAAGTCCGGCACCGCCTCCGTGGTGTGCCTGCTGAACAAC
TTCTACCCCCGCGAGGCCAAGGTGCAGTGGAAGGTGGACAACGCCCTG
CAGAGCGGCAACAGCCAGGAGAGCGTCACCGAGCAGGACAGCAAGGAC
TCCACCTACAGCCTGAGCAGCACCCTGACCCTGAGCAAGGCCGACTAC
GAGAAGCACAAGGTGTACGCCTGCGAGGTGACCCACCAGGGCCTGTCC
AGCCCCGTGACCAAGAGCTTCAACAGGGGCGAGTGCTGA

When age appropriate, the founder animals were bred. Following pregnancy and parturition the goats were milked. For example, Goat N0366 had been through two natural lactations producing approximately 1.5-2 L of milk per day for 200 days, resulting in 60-70 g/L of Herceptin/trastuzumab.

### Example 2: Herceptin/Trastuzumab Clarification

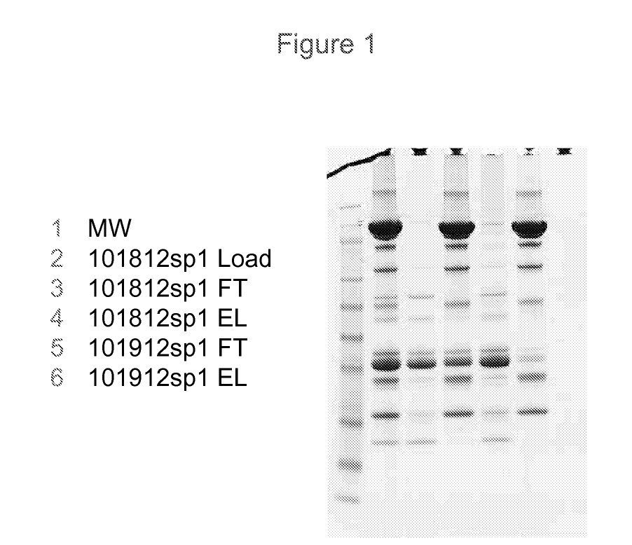

Milk that was harvested from transgenic goats was subjected to a clarification process. Briefly, the milk temperature was increased to 37° C., then the cream and skimmed milk were separated using a cream separator. Following removal of the cream, 1% Triton X100 was added to the milk and the mixture was incubated for 1 hour. The milk was then diluted to achieve an appropriate pH and conductivity and applied to a column containing sulfopropyl (SP)-Sepharose Fast Flow agarose beads. FIG. 1 shows a protein gel containing the flow through (FT) and elution (EL) from the Sepharose clarification column for two milk samples. The majority of the Herceptin/trastuzumab was present in the elution samples but not the flow through samples. Finally, the solution was passed through a 0.2 μm filter, resulting in a clarified intermediate with >50% purity.

### Example 3: Digestion and Purification of Herceptin/Trastuzumab

Clarified intermediate was subjected to Protein A affinity chromatography and applied to a column containing quaternary ammonium (Q)-Sepharose fast flow agarose beads. The flow through fraction from the Q-Sepharose fast flow column was concentrated and diafiltered into an appropriate digestion buffer including 20 mM phosphate pH 7.0 and 10 mM EDTA.

Various enzymes capable of digesting antibodies were tested for their utility in the present invention. Digestion reaction were prepared as shown in Table 1. Antibody digestion with papain was expected to result in an Fc fragment and two Fab fragments. Digestion with pepsin was expected to result in a single F(ab′)2 fragment and degraded Fc fragments. Digestion with ficin was expected to result in two Fab fragments and degraded Fc fragments. Similar to digestion with papain, digestion with trypsin was expected to result in an Fc fragment and two Fab fragments.

TABLE 1
Immobilized enzyme evaluation

| | | | Typical Time and |
|---|---|---|---|
| Enzyme | Buffer Conditions | Amt Enzyme | Temp |
| Papain | 20 mM phosphate pH 7.0 + | 0.25 mL/1 mL | 5-24 hours 37° C. |
| | 10 mM EDTA + 10 mM Cysteine- | | |
| | Herceptin #1 (UF/DF pool #071612q1-3): | | |
| | 2.2 mg/mL | | |
| Pepsin | 20 mM Na acetate pH 4.5- | 0.25 mL/1 mL | 2-4+ hours 37° C. |
| | Herceptin #2 (UF/DF pool #071612q1-3):2.3 mg/mL | | |
| Ficin | 0.1M citrate pH 6.0 + 5 mM | 0.25 mL/1 mL | 3-5 hours 37° C. |
| | EDTA + 25 mM Cysteine- | | |
| | Herceptin #3 (UF/DF pool #071612q1-3): | | |
| | 2.3 mg/ML | | |
| Trypsin | 50 mM tris pH 8.0 + 50 mM Cacl₂ + | 0.25 mL/1 mL | 3-5 hours 37° C. |
| | 10 mM cysteine- | | |
| | Herceptin #4 (UF/DF pool #071612q1-3): | | |
| | 2.3 mg/mL | | |

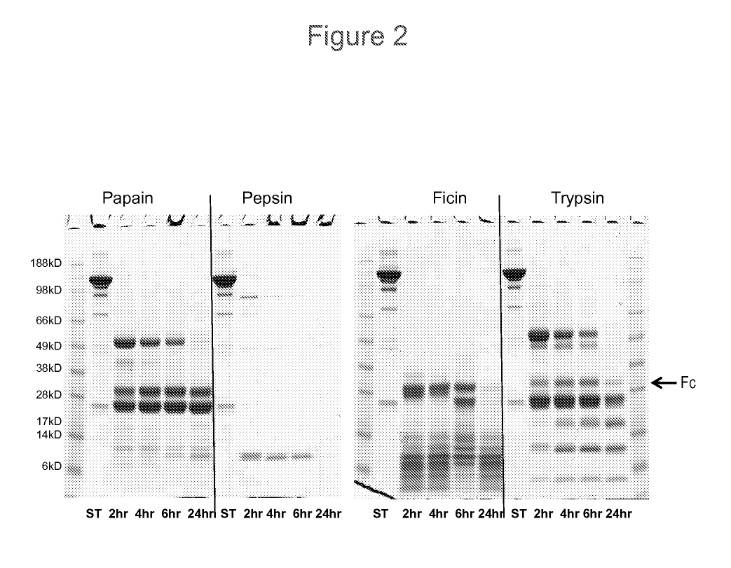

Following incubation for the indicated time, digestion reactions were evaluated by SDS-PAGE protein gel, as shown in FIG. 2. Papain was selected as the digestion enzyme for further development.

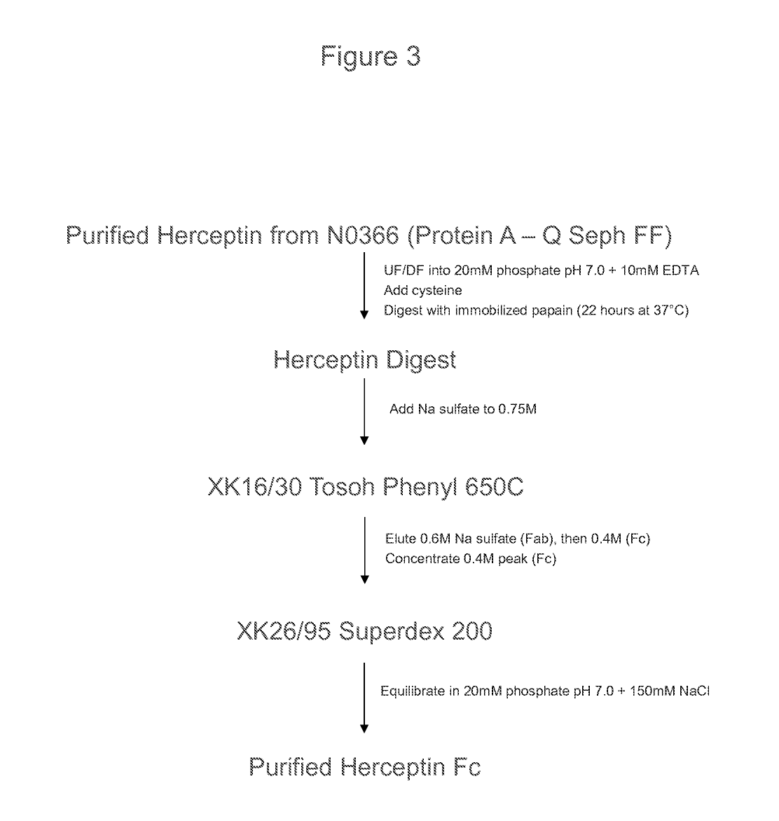

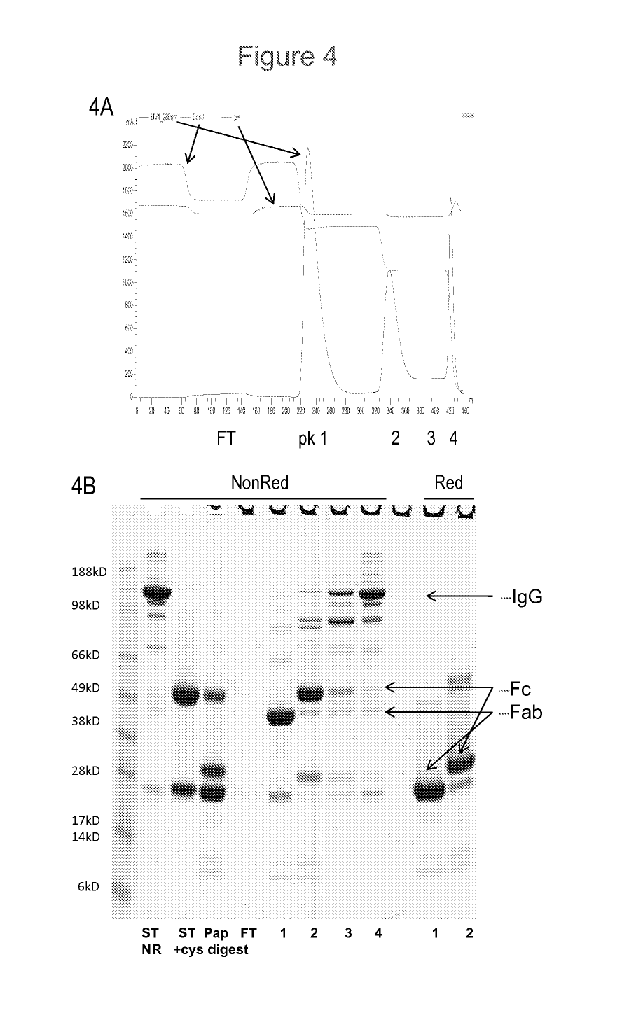

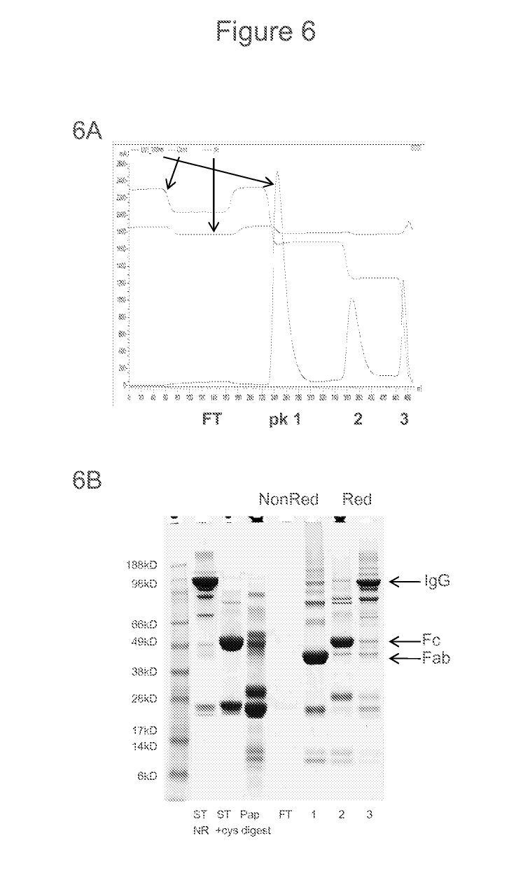

As outlined in the work flow of FIG. 3, transgenically-produced Herceptin/trastuzumab was clarified and purified by Protein A affinity chromatography and applied to a column containing Q-Sepharose Fast Flow agarose beads. Ultrafiltration/diafiltration was then performed to exchange the buffer to 20 mM phosphate pH 7.0, 10 mM EDTA. The antibody was digested with immobilized papain in the presence of cysteine for 22 hours at 37° C. After completion of the digestion, the papain was removed from the digested Herceptin/trastuzumab. Sodium sulfate was added to a concentration of 0.75 M, and the digested antibody was applied to a XK16/30 Tosoh Phenyl 650C hydrophobic interaction chromatography (HIC) column that had been equilibrated in 20 mM phosphate pH 7.0 with 1M sodium sulfate. The column was washed with 20 mM phosphate pH 7.0 (sodium sulfate concentration of 0 M) to remove any undigested antibody. The column was eluted using a decreasing concentration of sodium sulfate. Fab fragments eluted from the column at approximately 0.6M sodium sulfate, and Fc fragments eluted from the column at approximately 0.4M sodium sulfate. FIG. 4A and FIG. 6A depict HIC traces for two independent preparations of digested Herceptin/trastuzumab. FIG. 4B and FIG. 6B show SDS-PAGE protein gels of samples from each of the Hereceptin/trastuzumab preparations, including samples collected from each of the peaks indicated in the HIC traces (FIG. 4A and FIG. 6A). Table 2 summarizes the yield of Fc fragments in the indicated HIC peaks. Herceptin/trastuzumab preparation 081413ph1 (depicted in FIGS. 4A and 4B) resulted in 372 mg (92.1%) total protein recovery and 99 mg (26.6%) Fc fragment recovery, as calculated by A280 nm with an extinction coefficient of 1.4. Herceptin/trastuzumab preparation 082013phe1 resulted in 329 mg (81.4%) total protein recovery and 93 mg (28.3%) Fc fragment recovery. Herceptin/trastuzumab preparation 082113phe1 resulted in 332 mg (82.2%) total protein recovery and 96 mg (28.9%) Fc fragment recovery.

TABLE 2
Summary of Herceptin/trastuzumab Fc purification by
HIC after papain digest
Herceptin-Fc Purification Summary - Papain Digest 081313-082113

| | | #mg | mg/mL | | | | Total #mg | | | |
|---|---|---|---|---|---|---|---|---|---|---|
| Prep # | Column | start | pk 1 | 2 | 3 | 4 | pk 1 | 2 | 3 | 4 |
| 081413phe1 | Tosoh | 404 | 3.3 | 2.1 | 0.6 | 2.7 | 205 | 99 | 17 | 51 |
| | Phenyl | | | | | | | | | |
| 082013phe1 | Tosoh | 404 | 2.9 | 2.0 | 0.9 | 2.0 | 179 | 93 | 19 | 38 |
| | Phenyl | | | | | | | | | |
| 082113phe1 | Tosoh | 404 | 2.1 | 1.7 | 0.8 | 1.4 | 191 | 96 | 15 | 30 |
| | Phenyl | | | | | | | | | |

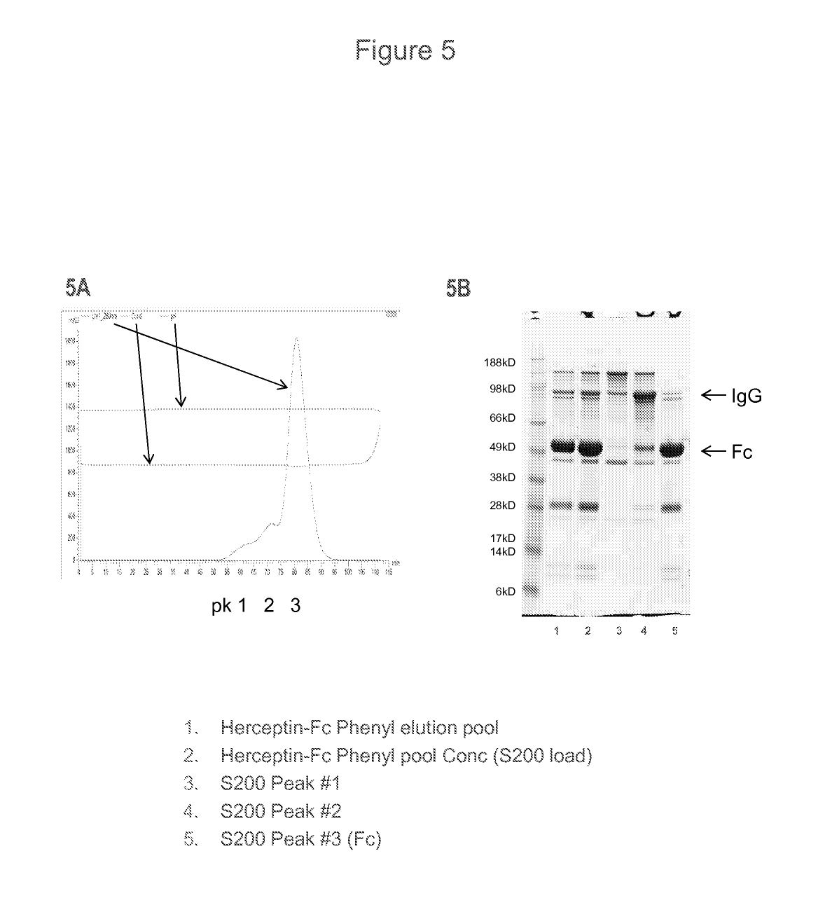

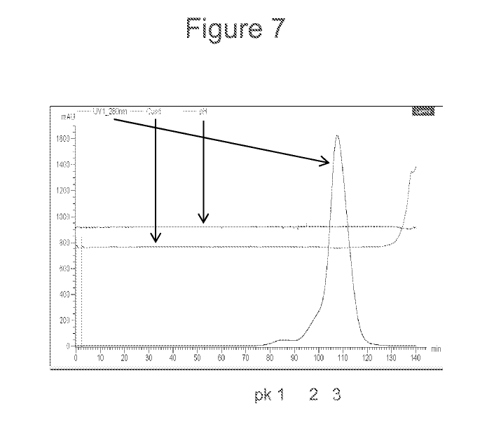

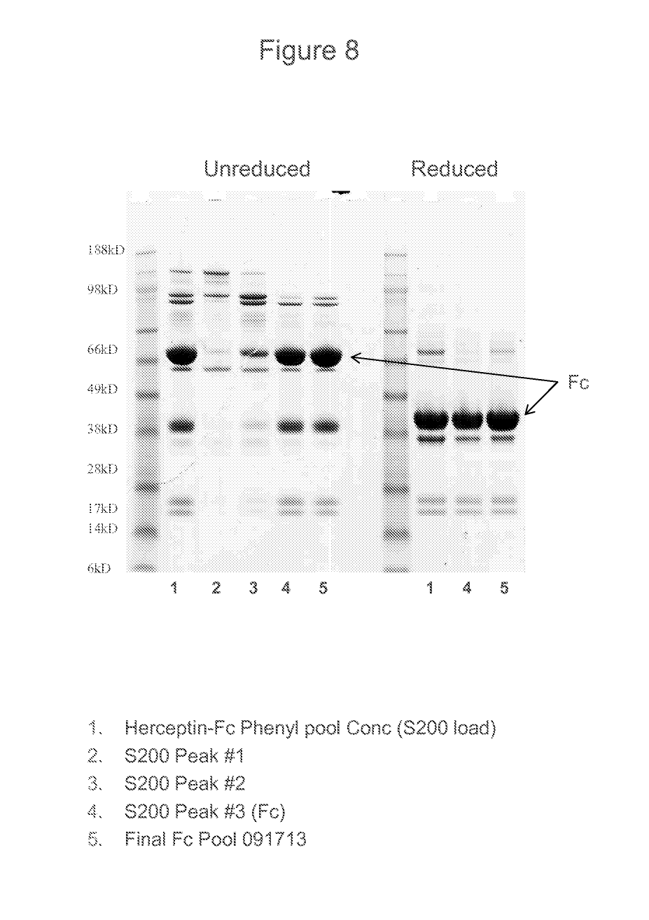

The eluate at 0.4M sodium sulfate (containing Fc fragments) were collected and concentrated then further purified by gel filtration chromatography using a XK26/95 Superdex 200 column (FIG. 5A and FIG. 7). The column was eluted with an isocratic run using 20 mM phosphate pH 7.0 and 150 mM NaCl. The collected samples from each of the peaks of the HIC trace were further analyzed by SDS-PAGE gel (FIGS. 5B and 8). Table 3 summarizes the yield of Fc fragments in the indicated peaks from the gel filtration chromatography. Herceptin/trastuzumab preparation 082613s200a resulted in 105 mg (92.9%) total protein recovery and 88 mg (83.8%) Fc fragment recovery. Herceptin/trastuzumab preparation 082713s200a resulted in 111 mg (98.2%) total protein recovery and 95 mg (85.6%) Fc fragment recovery. Herceptin/trastuzumab preparation 082713s200b resulted in 116 mg (102.3%) total protein recovery and 98 mg (84.5%) Fc fragment recovery.

TABLE 3
Summary of Herceptin/trastuzumab Fc purification by gel filtration

Pool the following and concentrate prior to gel filtration:
080813phe1 EL3 (95 mg)
081413phe1 EL2 (99 mg)
082013phe1 EL2 (93 mg)
082113phe1 EL2 (96 mg)
383 mg total
Concentrate to 25 mL (082613tff1, 13.6 mg/mL, 340 mg total, 88%
recovery)

| | | #mg | mg/mL | | | Total #mg | | |
|---|---|---|---|---|---|---|---|---|
| Prep # | Column | start | pk 1 | 2 | 3 | pk 1 | 2 | 3 |
| 082613s200a | Superdex | 113 | 0.34 | 0.98 | 3.40 | 7 | 11 | 88 |
| | 200 | | | | | | | |
| 082713s200a | Superdex | 113 | 0.35 | 0.95 | 3.29 | 6 | 10 | 95 |
| | 200 | | | | | | | |
| 082713s200b | Superdex | 113 | 0.65 | 0.96 | 3.65 | 7 | 11 | 98 |
| | 200 | | | | | | | |

Final Fc Pool: 84 mL @ 3.33 mg/mL (280 mg total)

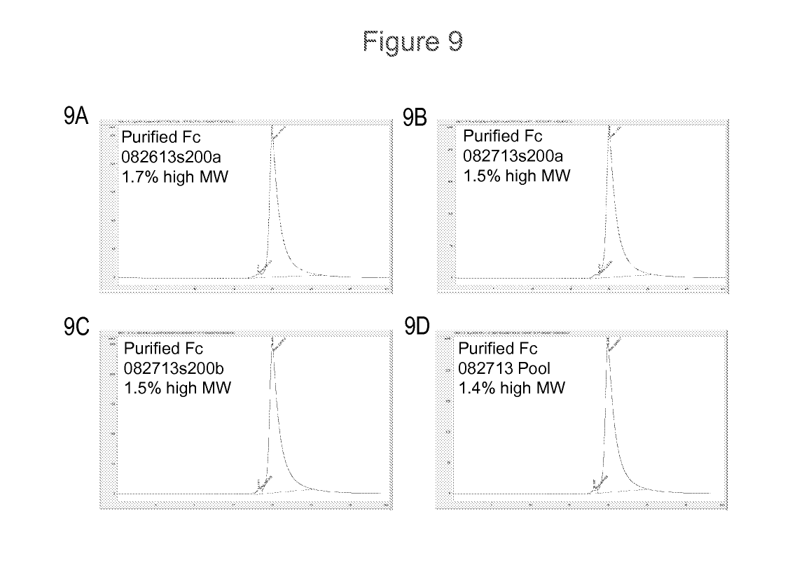

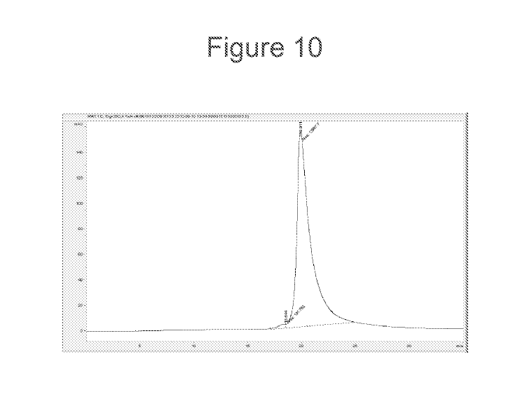

The samples containing Fc fragments were pooled to form final Fc pools (FIG. 8, sample number 5). The purity of the samples and the final pools was also assessed by HPLC-SEC (FIG. 9, FIG. 10).

What is claimed is:

1. A method of producing a fragment crystallizable (Fc) fragment, the method comprising
providing a transgenic non-human mammal that has been modified to express an antibody comprising an Fc fragment in the mammary gland;
harvesting the antibody comprising the Fc fragment from milk produced by the mammary gland of the transgenic mammal; and
isolating the Fc fragment from the antibody.

2. A method of producing an Fc fragment, the method comprising
providing a mammary epithelial cell that has been modified to express an antibody comprising an Fc fragment;
harvesting the antibody comprising the Fc fragment from the mammary epithelial cell; and
isolating the Fc fragment from the antibody.

3. A method of producing an Fc fragment, the method comprising
providing a transgenic non-human mammal that has been modified to express an Fc fragment in the mammary gland;
harvesting the Fc fragment from the milk produced by the mammary gland of the transgenic mammal; and
isolating the Fc fragment.

4. A method of producing an Fc fragment, the method comprising
providing a mammary epithelial cell that has been modified to express an Fc fragment;
harvesting the Fc fragment from the mammary epithelial cell; and
isolating the Fc fragment.

5. The method of claim 1 or 2, wherein isolating the Fc fragment comprises subjecting the antibody sequentially to
(a) hydrophobic interaction chromatography; and
(b) ultrafiltration.

6. The method of claim 3 or 4, wherein isolating the Fc fragment comprises subjecting the Fc fragment sequentially to
(a) hydrophobic interaction chromatography; and
(b) ultrafiltration.

7. The method of claim 5 or 6, wherein the ultrafiltration is performed in a solution comprising phosphate, NaCl and Tween 80, wherein the phosphate has a concentration between 10 and 100 mM, the NaCl has a concentration between 100 and 500 mM, and the Tween 80 has a concentration between 0 to 0.01%, optionally wherein the solution comprises 20 mM phosphate pH 7.0, 150 mM NaCl and 0.01% Tween 80.

8. The method of claim 1 or 2, wherein isolating the Fc fragment from the antibody comprises
(a) obtaining an antibody comprising an Fc fragment and one or more additional fragments;
(b) digesting the antibody of (a) to produce an Fc fragment and one or more additional fragments;
(c) separating the Fc fragment from the one or more additional fragments by hydrophobic interaction chromatography, comprising
applying the Fc fragment and the one or more additional fragments of (b) to a hydrophobic interaction chromatography column; and
recovering the Fc fragment from the hydrophobic interaction chromatography column; and
(d) further purifying the recovered Fc fragment by ultrafiltration.

9. The method of claim 8, wherein the one or more additional fragments include a fragment antigen-binding (Fab) fragment, a Fab′ fragment, a F(ab′)2 fragment or a single-chain variable (scFv) fragment.

10. The method of claim 3 or 4, wherein isolating the Fc fragment comprises
(a) obtaining an Fc fragment;
(b) subjecting the Fc fragment to hydrophobic interaction chromatography, comprising
applying the Fc fragment of (a) to a hydrophobic interaction chromatography column; and
recovering the Fc fragment from the hydrophobic interaction chromatography column; and
(c) further purifying the recovered Fc fragment by ultrafiltration.

11. The method of claim 2 or 4, wherein the mammary epithelial cell is of a non-human mammal.

12. The method of claim 1, 3 or 11, wherein the non-human mammal is a goat, sheep, bison, camel, cow, pig, rabbit, buffalo, horse, rat, mouse, or llama.

13. The method of claim 1 or 3, wherein the transgenic non-human mammal is also transgenic for the expression of a sialyl transferase.

14. The method of claim 8, wherein obtaining the antibody comprises purifying the antibody.

15. The method of claim 10, wherein obtaining the Fc fragment comprises purifying the Fc fragment.

16. The method of claim 14, wherein the antibody is purified using affinity chromatography.

17. The method of claim 15, wherein the Fc fragment is purified using affinity chromatography.

18. The method of claim 16 or 17, wherein the affinity chromatography comprises Protein A affinity chromatography.

19. A method comprising subjecting an antibody sequentially to
(a) hydrophobic interaction chromatography; and
(b) ultrafiltration,
wherein the ultrafiltration is performed in a solution comprising phosphate, NaCl and Tween 80, wherein the phosphate has a concentration between 10 and 100 mM, the NaCl has a concentration between 100 and 500 mM, and the Tween 80 has a concentration between 0 to 0.01%, optionally wherein the solution comprises 20 mM phosphate pH 7.0, 150 mM NaCl and 001% Tween 80.

20. The method of 19, wherein the antibody is digested prior to hydrophobic interaction chromatography.

21. The method of claim 8 or 20, wherein the digestion is performed by an enzyme.

22. The method of claim 21, wherein the enzyme is a cysteine protease.

23. The method of claim 22, wherein the cysteine protease is papain.

24. The method of claim 23, wherein the papain is immobilized on a solid support.

25. The method of any one of claims 1, 2, 5, 7-9, 11-14, 16, 18-24, wherein the antibody isotype is IgE, IgG, IgA, IgM or IgD.

26. The method of claim 25, wherein the antibody isotype is IgG.

27. The method of claim 26, wherein the antibody is Herceptin.

28. A method comprising subjecting an Fc fragment sequentially to
(a) hydrophobic interaction chromatography; and
(b) ultrafiltration,
wherein the ultrafiltration is performed in a solution comprising phosphate, NaCl and Tween 80, wherein the phosphate has a concentration between 10 and 100 mM, the NaCl has a concentration between 100 and 500 mM, and the Tween 80 has a concentration between 0 to 0.01%, optionally wherein the solution comprises 20 mM phosphate pH 7.0, 150 mM NaCl and 001% Tween 80.

29. The method of any one of claims 1-28, wherein the purity of the isolated Fc fragment is at least 95%, 96%, 97%, 98%, 99%, 99.5% or 99.9%.

30. The method of claim 29, wherein the purity of the isolated Fc fragment is assessed by high performance liquid chromatography, SDS-PAGE gel electrophoresis, or contaminant protein ELISA.

31. The method of any one of claims 5, 6, 8, 10, 19, 28, wherein the hydrophobic interaction chromatography is performed using a hydrophobic chromatography column comprising an organic polymer resin.

32. The method of claim 31, wherein the organic polymer resin is phenyl organic polymer resin.

33. The method of any one of claims 5, 6, 8, 10, 19, 28, wherein the hydrophobic interaction column is eluted using a salt buffer.

34. The method of claim 33, wherein the elution of the hydrophobic interaction column is performed using a decreasing gradient of the salt buffer concentration.

35. The method of any one of claims 5, 6, 8, 10, 19, 28, wherein ultrafiltration is performed using gel filtration chromatography.

36. The method of any one of claims 1-35, wherein the Fc fragment has anti-inflammatory properties.

37. The method of any one of claims 1-36, wherein the Fc fragment is used to treat a subject with an autoimmune condition or an inflammatory condition.

38. A purified Fc fragment produced by the method of any one of the preceding claims.

39. A method comprising administering a therapeutically effective amount of an Fc fragment produced in a transgenic non-human mammal to a subject in need thereof.

40. The method of claim 39, wherein the subject has an inflammatory condition or an autoimmune condition.

41. A transgenic Fc fragment.

42. The transgenic Fc fragment of claim 41, wherein the transgenic Fc fragment is purified.

43. A method comprising administering a therapeutically effective amount of the transgenic Fc fragment of claim 41 or 42 to a subject in need thereof.

44. The method of claim 43, wherein the subject has an inflammatory condition or an autoimmune condition.


SEQUENCE LISTING (5 sequences)

SEQ ID NO: 1 - protein, 217 aa, artificial sequence
  synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 469 aa, artificial sequence
  synthetic polypeptide
MEFGLSWLFLVAILKGVQCEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 236 aa, artificial sequence
  synthetic polypeptide
MDMRVPAQLLGLLLLWLRGARCDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - DNA, 1413 bp, artificial sequence
  synthetic polynucleotide
atggagttcggcctgagctggctgttcctggtggccatcctgaagggcgtgcagtgcgaggtgcagctggtcgagagcggaggaggactggtccagcctggcggcagcctgagactgagctgcgccgccagcggcttcaacatcaaggacacctacatccactgggtgcgccaggctccagggaaagggctcgaatgggtggccaggatctaccccaccaacggctacaccagatacgccgacagcgtgaagggcaggttcaccatcagcgccgacaccagcaagaacaccgcctacctgcagatgaacagcctgagggccgaggacaccgccgtgtactactgcagcagatggggtggggatggcttctacgccatggactactgggggcagggcacactggtcacagtctccagcgccagcaccaagggccccagcgtgttccccctggctccttcctctaaatccacaagcggcggcaccgctgccctgggctgcctggtgaaggactacttccccgagcccgtgaccgtgtcttggaactctggcgccctgacctccggcgtgcacaccttccccgccgtgctgcagagcagcggcctgtacagcctgagcagcgtggtgaccgtgccctcttcctctctcggaacacagacctacatctgcaacgtgaaccacaagcccagcaacaccaaggtggacaagaaggtggagcccaagagctgcgacaagacccatacatgtcctccctgtcctgctcctgagctgctgggcggaccctccgtgttcctgttcccccccaagcccaaggacaccctgatgatcagcaggacccccgaggtgacctgcgtggtggtggacgtgtcccacgaggaccctgaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagcccccatcgaaaagaccatcagcaaggccaagggccagcctcgcgagccccaggtgtacaccctgcccccctcccgcgacgagctgaccaagaaccaggtgtccctgacctgtctggtgaagggcttctaccccagcgatatcgccgtggagtgggagagcaacggccagcccgagaacaactacaagaccaccccccctgtgctggacagcgacggcagcttcttcctgtacagcaagctgaccgtggacaagagcaggtggcagcagggaaatgtcttttcctgttccgtcatgcatgaagctctgcacaaccactacacccagaagtccctgagcctgagccccggcaagtgatag

SEQ ID NO: 5 - DNA, 711 bp, artificial sequence
  synthetic polynucleotide
atggacatgagagtgcctgcccagctcctgggactcctcctcctgtggctcaggggtgctcgctgcgatatccagatgactcagtctccttcttccctctccgccagcgtgggcgacagagtgaccatcacctgcagggccagccaggacgtgaacaccgccgtggcctggtatcagcagaagcccggcaaggcccccaagctgctgatctacagcgccagcttcctgtacagcggcgtgcccagcaggttcagcggcagcagaagcggcaccgacttcaccctgaccatcagcagcctgcagcccgaggacttcgccacctactactgccagcagcactacaccaccccccccaccttcggccagggcaccaaggtggagatcaagaggaccgtggccgctcccagcgtgttcatcttcccccccagcgacgagcagctgaagtccggcaccgcctccgtggtgtgcctgctgaacaacttctacccccgcgaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaacaggggcgagtgctga